(19) United States
(12) Patent Application Publication
(10) Pub. No.: US 2025/0061567 A1
(43) Pub. Date: Feb. 20, 2025

(54) MEDICAL EQUIPMENT FOR DIAGNOSING AND MANAGING WOUNDS, AND MEDICAL EQUIPMENT OPERATION METHOD

(71) Applicant: FINEHEALTHCARE, Seoul (KR)

(72) Inventors: Hyun Kyung SHIN, Seoul (KR); Sang Hyuk OH, Seoul (KR); Hyeon Ung SHIN, Seoul (KR); Hyung Lae LEE, Seoul (KR); Byung Hyun MIN, Yongin-si Gyeonggi-do (KR); Eui Yul KOH, Seoul (KR); Hyun Gyu KIM, Seoul (KR)

(73) Assignee: FINEHEALTHCARE, Seoul (KR)

(21) Appl. No.: 18/718,318

(22) PCT Filed: Dec. 8, 2021
(86) PCT No.: PCT/KR2021/018494

§ 371 Date: Jul. 18, 2024

(51) Int. Cl. G06T 7/00; G06T 7/73; G06T 7/90; G06T 11/00

(52) U.S. Cl. CPC G06T 7/0012; G06T 7/74; G06T 7/90; G06T 11/001; G06T 2207/10024; G06T 2207/30088; G06T 2207/30204; G06T 2210/41

(57) ABSTRACT

The present disclosure relates to an apparatus for managing a wound and a method of operating an apparatus for managing a wound. The method includes acquiring a measured image including a color chart, acquiring a location of a first marker included in the color chart based on the measured image, acquiring a location of a second marker included in the color chart based on the measured image, acquiring a location of a third marker included in the color chart based on the measured image, acquiring a location of a fourth marker included in the color chart based on the measured image, and acquiring center points of a plurality of color patches included in the color chart based on the location of the first marker, the location of the second marker, the location of the third marker, and the location of the fourth marker.

### TECHNICAL FIELD

The present disclosure relates to an apparatus for managing a wound and a method of operating the apparatus for managing a wound. More particularly, the wound management device of the present disclosure may accurately correct an image containing a wound.

### BACKGROUND ART

Depending on the cause, wounds are classified into an incised wound, a puncture wound, a chop wound, an acne wound, a fissure, a firearm wound, and a bite wound, and depending on the shape, wounds are classified into a linear wound, a valvular wound, and a defective wound. In addition, wounds are divided into a penetrating wound and a non-penetrating wound based on the relationship with a body cavity, and into a septic wound and a non-septic wound based on the presence or absence of infection. Symptoms of wounds include pain, bleeding, dysfunction, etc. When injured, a living body reacts immediately and tries to recover quickly from the injury (see Doosan Encyclopedia).

A treatment process varies depending on general conditions of the living body or the wounds, but usually, wounds are treated according to the following process: \textcircled{1} Degeneration and death of injured cells, \textcircled{2} Outflow of pericyte tissue fluid from surrounding tissues, \textcircled{3} Precipitation of fibrin and formation of granulation tissue, etc.

In order to treat wounds quickly, treatment methods according to the wounds should be applied, but due to the patient's lack of knowledge about wound treatment, the treatment of wounds progresses slowly or scars may occur. For example, iodine tincture or mercurochrome, called red medicine, is generally mainly used for treating wounds. The iodine tincture or mercurochrome is an antibacterial agent, and serves to kill germs. However, even though these drugs have a sterilizing effect, they actually interfere with regenerating epithelial cells or fibroblasts in the process of treating most wounds, resulting in slowing down the treatment process. Therefore, there is a need for an apparatus for providing information on a correct treatment method depending on the wounds.

### DISCLOSURE

### Technical Solution

An apparatus for managing a wound according to an embodiment of the present disclosure may diagnose and manage a wound by capturing a color chart. A method of operating an apparatus for managing a wound according to an embodiment of the present disclosure includes acquiring a measured image including a color chart, acquiring a location of a first marker included in the color chart based on the measured image, acquiring a location of a second marker included in the color chart based on the measured image, acquiring a location of a third marker included in the color chart based on the measured image, acquiring a location of a fourth marker included in the color chart based on the measured image, and acquiring center points of a plurality of color patches included in the color chart based on the location of the first marker, the location of the second marker, the location of the third marker, and the location of the fourth marker.

The first marker may be located at an upper left of the color chart, the second marker may be located at an upper right of the color chart, the third marker may be located at a lower left of the color chart, and the fourth marker may be located at a lower right of the color chart.

The acquiring of the center points of the plurality of color patches may include generating a first horizontal line connecting the location of the first marker and the location of the second marker, and determining n−1 points by dividing the first horizontal line into n equal parts to be the center points of the plurality of color patches. The acquiring of the center points of the plurality of color patches may include generating a first vertical line connecting the location of the first marker and the location of the third marker, generating a second vertical line connecting the location of the second marker and the location of the fourth marker, determining a first sub point by bisecting the first vertical line, determining a second sub point by bisecting the second vertical line, generating a second horizontal line connecting the first sub point and the second sub point, and determining m−1 points by dividing the second horizontal line into m equal parts to be the center points of the plurality of color patches.

The acquiring of the center points of the plurality of color patches may include generating a third vertical line connecting the location of the third marker and the location of the fourth marker, and determining k−1 points by dividing the third horizontal line into k equal parts to be the center points of the plurality of color patches.

The method of operating an apparatus for managing a wound may further include acquiring a first pixel length by dividing a pixel length of the first horizontal line into n equal parts, acquiring a second pixel length by dividing a pixel length of the second horizontal line into m equal parts, acquiring a third pixel length by dividing a pixel length of the third horizontal line into k equal parts, determining a representative pixel length using at least one of the first pixel length, the second pixel length, and the third pixel length, acquiring a predetermined actual distance between the center points of the plurality of color patches, and acquiring an actual distance per pixel using the predetermined actual distance and the representative pixel length.

The method of operating an apparatus for managing a wound may further include determining a seventh point, an eighth point, and a ninth point by quartering the third horizontal line to be the center points of the plurality of color patches, acquiring the center points of the plurality of color patches, and then correcting a red color of the measured image based on a pixel value of the seventh point, correcting a green color of the measured image based on a pixel value of the eighth point, and correcting a blue color of the measured image based on a pixel value of the ninth point.

The plurality of color patches included in the color chart may each be one of squares whose one-side lengths are 5 mm, 10 mm, 15 mm, and 20 mm, and a distance between the plurality of color patches is 2 mm.

The acquiring of the measured image including the color chart may include acquiring a preview image including the color chart, acquiring the location of the first marker included in the color chart based on the preview image, acquiring the location of the second marker included in the color chart based on the preview image, acquiring the location of the third marker included in the color chart based on the preview image, acquiring the location of the fourth marker included in the color chart based on the preview image, determining whether a first difference value between a y-axis value of the location of the first marker and a y-axis value of the location of the second marker is within a critical distance, determining whether a second difference value between a y-axis value of the location of the third marker and a y-axis value of the location of the fourth marker is within the critical distance, determining whether a third difference value between an x-axis value of the location of the first marker and an x-axis value of the location of the third marker is within the critical distance, determining whether a fourth difference value between an x-axis value of the location of the second marker and an x-axis value of the location of the fourth marker is within the critical distance, and when the first difference value, the second difference value, the third difference value, and the fourth difference value are all within the critical distance, acquiring the preview image as the measured image.

The method of operating an apparatus for managing a wound may further include displaying a predetermined icon at the location of the first marker on the preview image, displaying a fourth horizontal line on the preview image, which is parallel to a horizontal axis of the preview image and passes through the location of the first marker, on the preview image, displaying a third vertical line on the preview image, which is parallel to a vertical axis of the preview image and passes through the location of the first marker, on the preview image, and displaying predetermined icons at the location of the second marker, the location of the third marker, and the location of the fourth marker on the preview image.

In addition, a program for implementing the method of operating an apparatus for managing a wound as described above may be recorded on a computer-readable recording medium.

### DESCRIPTION OF DRAWINGS

FIG. 1 is a diagram illustrating an apparatus for managing a wound according to an embodiment of the present disclosure.

FIG. 2 is a flowchart illustrating an operation of the apparatus for managing a wound according to an embodiment of the present disclosure.

FIG. 3 is a diagram illustrating a color chart according to an embodiment of the present disclosure.

FIG. 4 is a diagram for describing an operation of an apparatus for managing a wound according to an embodiment of the present disclosure.

FIG. 5 is a table for describing a predetermined actual distance between center points of color patches according to an embodiment of the present disclosure.

FIG. 6 is a diagram for describing an image acquisition unit according to an embodiment of the present disclosure.

FIG. 7 is a diagram illustrating a color chart according to an embodiment of the present disclosure.

FIG. 8 is a diagram illustrating color charts of a plurality of sizes according to an embodiment of the present disclosure.

### BEST MODE

Various advantages and features of disclosed embodiments and methods accomplishing them will become apparent from the following description of embodiments with reference to the accompanying drawings. However, the present disclosure is limited to exemplary embodiments disclosed below, but may be implemented in various different forms. These embodiments will be provided only in order to make the disclosure of the present disclosure complete and allow those skilled in the art to which the present disclosure pertains to completely recognize the scope of the present disclosure.

After terms used in the present specification are briefly described, the present disclosure will be described in detail.

General terms that are currently widely used are selected as terms used in embodiments in consideration of functions in the present specification but may be changed depending on the intention of those skilled in the art or a judicial precedent, the emergence of a new technique, and the like. In addition, in a specific case, terms arbitrarily chosen by an applicant may exist. In this case, the meaning of such terms will be mentioned in detail in a corresponding description portion of the present disclosure. Therefore, the terms used in embodiments of the disclosure are to be defined on the basis of the meaning of the terms and the contents throughout the disclosure rather than simple names of the terms.

In the present specification, singular forms include plural forms unless the context clearly indicates otherwise. In addition, plural forms include singular forms unless the context clearly indicates otherwise.

Throughout the specification, unless otherwise specified, “including” any component means that other components may be further included rather than excluding other components.

Also, as used herein, the term “unit” refers to a software or hardware component, and “unit” performs certain roles. However, the “unit” is not meant to be limited to software or hardware. The “unit” may be stored in a storage medium that can be addressed or may be configured to regenerate one or more processors. Accordingly, as an example, the “unit” refers to components such as software components, object-oriented software components, class components, and task components, processes, functions, attributes, procedures, subroutines, segments of program code, drivers, firmware, microcode, circuits, data, databases, data structures, tables, arrays and variables. Components and functions provided within “unit” may be combined into a smaller number of components and “unit” or may be further separated into additional components and “unit.”

According to an embodiment of the present disclosure, the “unit” may be implemented as a processor and a memory. The term “processor” should be interpreted broadly to include a general purpose processor, a central processing unit (CPU), a microprocessor, a digital signal processor (DSP), a controller, a microcontroller, a state machine, and the like. In some contexts, the “processor” may be an application specific semiconductor (ASIC), a programmable logic device (PLD), a field programmable gate array (FPGA), or the like. The term “processor” may also be a combination of processing devices, such as a combination of a DSP and a microprocessor, a combination of a plurality of microprocessors, a combination of one or more microprocessors in combination with a DSP core, or any other such configurations.

The term “memory” should be interpreted broadly to include any electronic component capable of storing electronic information. The term “memory” may mean any type of processor-readable medium such as a random access memory (RAM), a read-only memory (ROM), a non-volatile random access memory (NVRAM), a programmable read-only memory (PROM), an erase-programmable read-only memory (EPROM), an electrically erasable PROM (EEPROM), a flash memory, a magnetic or optical data storage device, or a register. The memory is said to be in electronic communication with a processor when the processor is capable of reading and/or writing information from and/or to the memory. The memory integrated in the processor is in electronic communication with the processor.

Hereinafter, embodiments of the disclosure will be described in detail with reference to the accompanying drawings so that those skilled in the art to which the present disclosure pertains may easily practice the present disclosure. In the drawings, parts irrelevant to the description are omitted in order to clarify the description of the present disclosure.

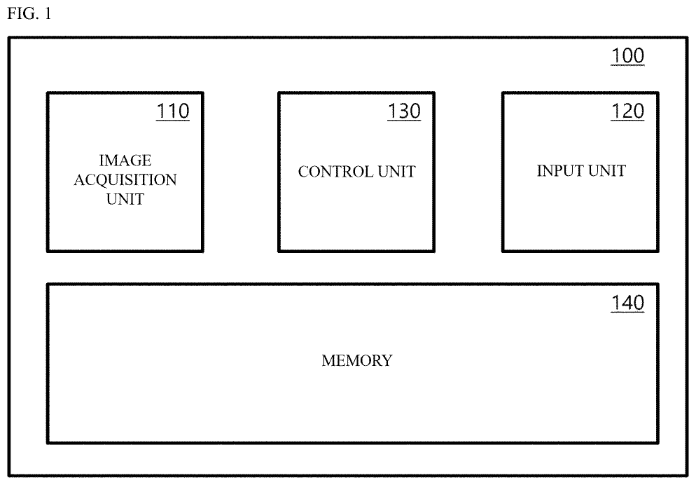

FIG. 1 is a diagram illustrating an apparatus for managing a wound according to an embodiment of the present disclosure.

An apparatus 100 for managing a wound may include an image acquisition unit 110, a control unit 130, an input unit 120, and a memory 140.

The image acquisition unit 110 may be an image sensor. The image acquisition unit 110 may convert light input through a lens into image data which is a digital signal. Image data may be stored in the memory 140.

The control unit 130 may be configured to control an operation of the apparatus 100 for managing a wound. The control unit 130 may be a processor. The control unit 130 may analyze image data acquired by the image acquisition unit 110.

The input unit 120 is a device for a user to input the image data to the apparatus 100 for managing a wound. For example, the input unit 120 may include at least one of a keyboard, a mouse, a touch screen, a digital pen, and a trackball.

The memory 140 is configured to store various types of data. In addition, the memory 140 may store commands for the control unit 130 to operate.

The apparatus 100 for managing a wound may further include a communication unit. The communication unit may be configured to communicate with an external device. The apparatus 100 for managing a wound may receive various types of data from the external device through the communication unit. For example, a database of the apparatus 100 for managing a wound may be configured in the memory 140, but may be configured in the external device. The apparatus 100 for managing a wound may store data in a database of the external device or receive data from the database.

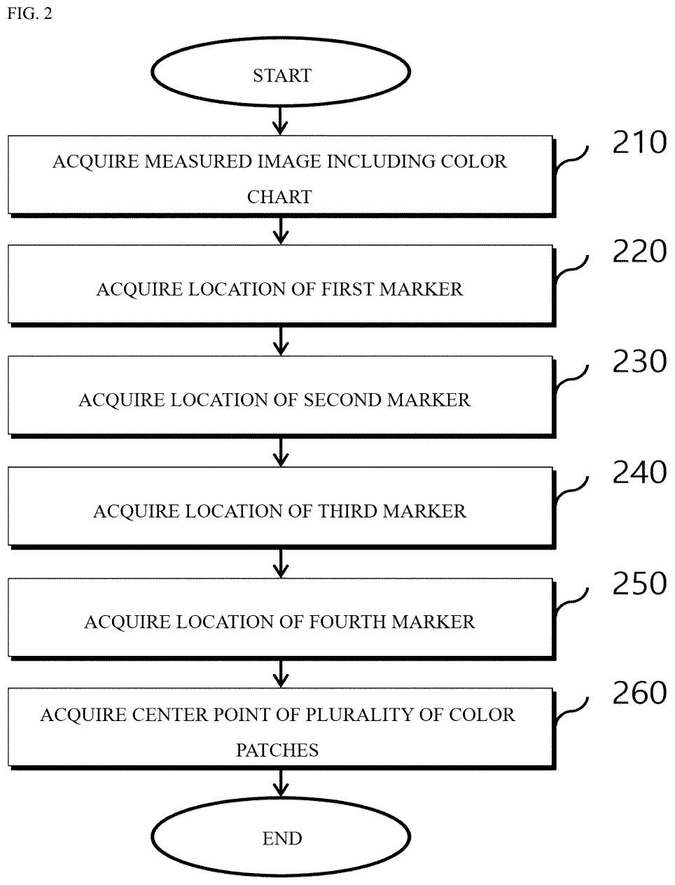

The apparatus 100 for managing a wound may include an output unit. The output unit may output various pieces of information as sound or light based on a signal from the control unit 130. The output unit may be at least one of a speaker and a display device. Hereinafter, the apparatus 100 for managing a wound will be described in more detail with reference to FIG. 2.

FIG. 2 is a flowchart illustrating an operation of the apparatus for managing a wound according to an embodiment of the present disclosure.

The apparatus 100 for managing a wound may collect various pieces of information related to a wound by capturing a color chart. In the present disclosure, the wound may be a bedsore or a burn.

The image acquisition unit 110 of the apparatus 100 for managing a wound may perform an operation 210 of acquiring a measured image including the color chart. The apparatus 100 for managing a wound may display the measured image including the color chart on the display unit through the image acquisition unit 110. The measured image including the color chart may include an image of a patient's wounded area. The apparatus 100 for managing a wound may use the output unit to output a message to capture an image so that the measured image including the color chart is input to a specific area of the display unit. The specific area may be a center of the display included in the apparatus 100 for managing a wound. In addition, the specific area may be an area selected by a user on the touch display of the apparatus 100 for managing a wound. The user may use the apparatus 100 for managing a wound to capture the measured image including the color chart while arranging an image of the wound in a specific area of a screen.

The apparatus 100 for managing a wound may perform the following operations to perform the operation 210 of acquiring the measured image including the color chart. The apparatus 100 for managing a wound may perform an operation of acquiring a preview image including the color chart. The preview image is an image displayed on the display of the apparatus 100 for managing a wound, and may be an image that changes in real time for a user to confirm an image to be captured before capturing the measured image. The apparatus 100 for managing a wound may determine the preview image to be the measured image when the preview image satisfies certain conditions. In other words, the apparatus 100 for managing a wound may automatically acquire the measured image when the preview image satisfies the measurement conditions.

The apparatus 100 for managing a wound may perform an operation of acquiring a location 411 of a first marker 321 included in a color chart 300 based on the preview image. In addition, the apparatus 100 for managing a wound may perform an operation of acquiring a location 412 of a second marker 322 included in the color chart 300 based on the preview image. The apparatus 100 for managing a wound may perform an operation of acquiring a location 413 of a third marker 323 included in the color chart 300 based on the preview image. The apparatus 100 for managing a wound may perform an operation of acquiring a location 414 of a fourth marker 324 included in the color chart 300 based on the preview image. The first to fourth markers 321 to 324 may be black and white symbols. Geometric shapes may be marked on the first to fourth markers 321 to 324.

The apparatus 100 for managing a wound may perform an operation of determining whether a first difference value between a y-axis value of the location 411 of the first marker 321 and a y-axis value of the location 412 of the second marker 322 is within a critical distance. The first difference value may be an absolute value. The critical distance may be a predetermined value. The critical distance may be, for example, 5 pixels.

The apparatus 100 for managing a wound may perform an operation of determining whether a second difference value between a y-axis value of the location 413 of the third marker 323 and a y-axis value of the location 414 of the fourth marker 324 is within the threshold distance. The apparatus 100 for managing a wound may perform an operation of determining whether a third difference value between an x-axis value of the location 411 of the first marker 321 and an x-axis value of the location 413 of the third marker 323 is within the critical distance. The apparatus 100 for managing a wound may perform an operation of determining whether a fourth difference value between an x-axis value of the location 412 of the second marker 322 and an x-axis value of the location 414 of the fourth marker 324 is within the critical distance.

The apparatus 100 for managing a wound may perform the operation of acquiring the preview image as the measured image when the first difference value, the second difference value, the third difference value, and the fourth difference value are all within the critical distance. The apparatus 100 for managing a wound may match a coordinate axis of the measured image and a coordinate axis of the color chart 300. In other words, the apparatus 100 for managing a wound may almost match an x-axis of the measured image and a horizontal axis of the color chart 300. In addition, the apparatus 100 for managing a wound may almost match a y-axis of the measured image and a vertical axis of the color chart 300.

The apparatus 100 for managing a wound may perform the following process to match the coordinate axis of the measured image and the coordinate axis of the color chart 300. The apparatus 100 for managing a wound may perform an operation of displaying a predetermined icon at the location 411 of the first marker 321 on the preview image. The icon may be a polygon such as a circle, a triangle, or a quadrangle. The icon may have a different color from a plurality of color patches 310 or the plurality of markers 321, 322, 323, and 324.

The apparatus 100 for managing a wound may perform an operation of displaying a fourth horizontal line, which is parallel to the horizontal axis of the preview image and passes through the location 411 of the first marker 321, on the preview image. The apparatus 100 for managing a wound may perform an operation of displaying a third vertical line, which is parallel to the vertical axis of the preview image and passes through the location 411 of the first marker 321, on the preview image.

The apparatus 100 for managing a wound may perform an operation of displaying a predetermined icon at least one of the location 412 of the second marker 322, the location 413 of the third marker 323, and the location 414 of the fourth marker 324 on the preview image.

The user may capture the measured image based on at least one of the fourth horizontal line, the third vertical line, the location 412 of the second marker 322, the location 413 of the third marker 323, and the location 414 of the fourth marker 324. In addition, when the location 412 of the second marker 322 is located on the fourth horizontal line, the apparatus 100 for managing a wound may automatically acquire the preview image as the measured image. In addition, when the location 413 of the third marker 323 is located on the third vertical line, the apparatus 100 for managing a wound may automatically acquire the preview image as the measured image. In addition, when the location 412 of the second marker 322 is located on the fourth horizontal line and the location 413 of the third marker 323 is located on the third vertical line, the apparatus 100 for managing a wound may automatically acquire the preview image as the measured image.

The apparatus 100 for managing a wound may perform an operation of displaying a fifth horizontal line, which is parallel to the horizontal axis of the preview image and passes through the location 414 of the fourth marker 324, on the preview image. The apparatus 100 for managing a wound may perform an operation of displaying a fourth vertical line, which is parallel to the vertical axis of the preview image and passes through the location 414 of the fourth marker 324, on the preview image.

When the location 412 of the second marker 322 is located at an intersection of the fourth horizontal line and the fourth vertical line, or the location 413 of the third marker 323 is located at an intersection of the fifth horizontal line and the third vertical line, the apparatus 100 for managing a wound may automatically acquire the preview image as the measured image.

The user may capture a wound image using the apparatus 100 for managing a wound while not being parallel to a wounded area. That is, in a state where a surface of a user's skin is not parallel to a surface facing the user in the apparatus 100 for managing a wound, the apparatus 100 for managing a wound may capture the wound image. In addition, in the state where the surface of color chart 300 is not parallel to the surface facing the user in the apparatus 100 for managing a wound, the apparatus 100 for managing a wound may capture the wound image. In this case, the wound image may appear reduced or stretched in the horizontal or vertical direction compared to the actual wound. However, when the preview image is acquired as the measured image using the above method, the surface of the color chart 300 may be parallel to the side facing the user in the apparatus 100 for managing a wound.

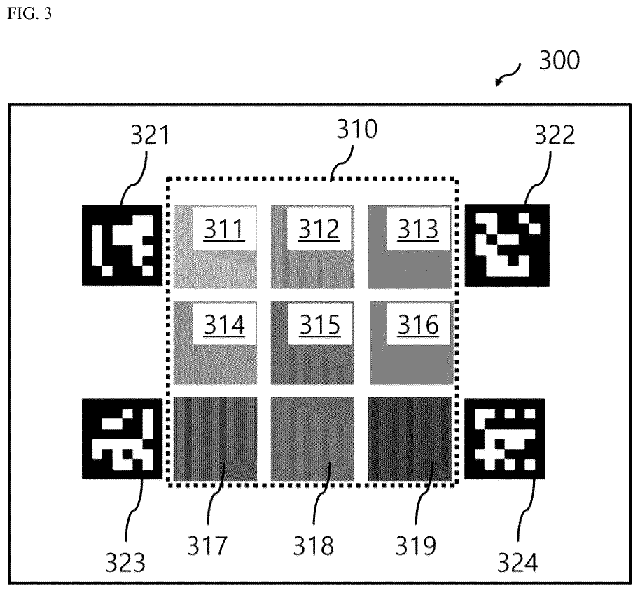

A color chart will be described below with reference to FIG. 3.

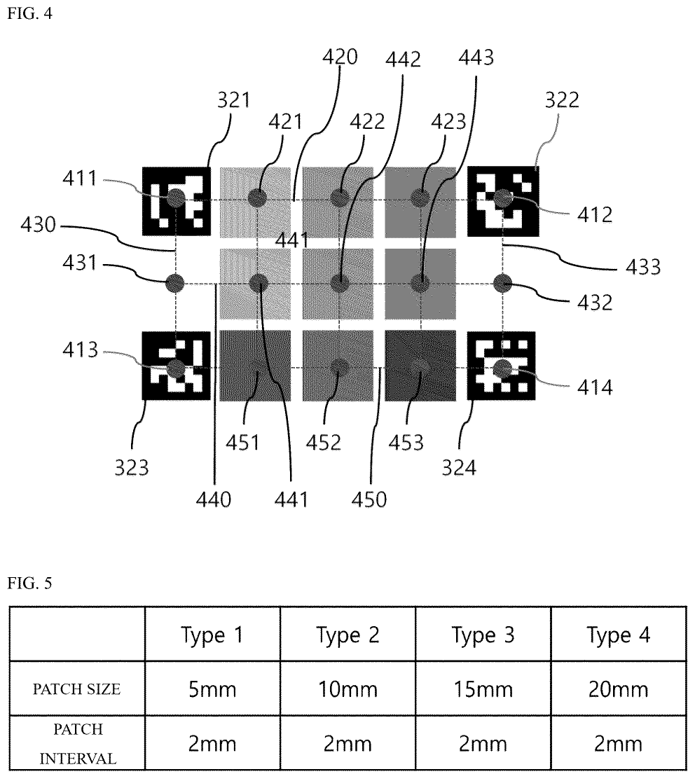

FIG. 3 is a diagram illustrating the color chart according to an embodiment of the present disclosure. FIG. 4 is a diagram illustrating the color chart according to an embodiment of the present disclosure. FIG. 7 is a diagram illustrating the color chart according to an embodiment of the present disclosure.

The apparatus 100 for managing a wound may automatically generate the color chart 300. For example, the apparatus 100 for managing a wound may acquire the image of the color chart 300 pre-stored in the memory and print the acquired image. In addition, the apparatus 100 for managing a wound may generate a new image of the color chart 300 based on at least one of an image obtained by capturing the user's skin or information related to a wound and print the generated image. However, it is not limited thereto, and a user may use the color chart 300 provided from a service provider.

The color chart 300 may include a plurality of figures painted in different colors. In the present disclosure, the plurality of figures painted in different colors included in the color chart 300 are referred to as a plurality of color patches. The apparatus 100 for managing a wound may determine a plurality of color patches 310 of a plurality of different colors to be included in the color chart 300 based on the type of wound. For example, the apparatus 100 for managing a wound may determine the color patches 310 having different colors to be included in the color chart 300 depending on whether the wound is a burn, a stab, or a bedsore. In addition, the apparatus 100 for managing a wound may determine the color patches 310 having a plurality of different colors to be included in the color chart 300 based on the time that has elapsed since the wound occurred. For example, the apparatus 100 for managing a wound may determine the color patches 310 having different colors to be included in the color chart 300 after 1 week, 2 weeks, or 1 month immediately after the wound occurred.

In addition, the apparatus 100 for managing a wound may determine the color patches 310 having different colors to be included in the color chart 300 based on a patient's skin color. The apparatus 100 for managing a wound may acquire an image of a patient's skin without a wound around the wound based on the image acquisition unit 110. In addition, the apparatus 100 for managing a wound may determine colors to be included in the color chart 300 based on the patient's skin without a wound. The apparatus 100 for managing a wound may blur the image of the patient's skin. The apparatus 100 for managing a wound may acquire colors of some of the images of the patient's skin after blurring the image of the patient's skin. The apparatus 100 for managing a wound may determine colors of a color patch 311 located on the upper left among the color patches 310 of the plurality of colors to be the colors of some of the blurred images of the patient's skin.

The apparatus 100 for managing a wound may enable the color chart 300 to include colors that mainly correspond to the skin tones of people around the world. For example, referring to FIG. 3, RGB values of the first color patch 311 may be 230, 202, and 183. RGB values of a second color patch 312 may be 199, 168, and 145. RGB values of a third color patch 313 may be 199, 159, and 145. RGB values of a fourth color patch 314 may be 199, 161, and 146. RGB values of a fifth color patch 315 may be 163, 123, and 94. RGB values of a sixth color patch 316 may be 195, 162, and 141. RGB values of a seventh color patch 317 may be 176, 62, and 60 or 255, 0, and 0. RGB values of an eighth color patch 318 may be 77, 142, and 68 or 0, 255, and 0. RGB values of a ninth color patch 319 may be 42, 76, and 149 or 0, 0, and 255. In FIG. 3, nine colors are presented, but the number of colors is not limited thereto. The color chart 300 may include colors less than 9 or exceeding 9 colors. For example, as illustrated in FIG. 7, the color chart 300 may include color patches of 11 colors. Hereinafter, the description is often made with reference to FIG. 3, but the same description may be applied to FIG. 7 as well.

In addition, the apparatus 100 for managing a wound may enable the color chart 300 to include main colors that appear during the treatment of a wound. For example, the color patches 310 included in the color chart 300 may include at least one of representative colors of blood, representative colors of pus, representative colors of scab, and representative colors of blisters. The apparatus 100 for managing a wound may determine main colors that appear in the process of treating the wound to be colors of color patches 711 to 718.

For example, referring to FIG. 7, the apparatus 100 for managing a wound may enable the color chart 300 to include colors that mainly appear in the wound. For example, RGB values of the color patch 711 may be 220, 188, and 165. RGB values of the color patch 712 may be 185, 123, and 86. RGB values of the color patch 713 may be 136, 62, and 51. RGB values of the color patch 714 may be 103, 56, and 38. RGB values of the color patch 715 may be 212, 161, and 157. RGB values of the color patch 716 may be 192, 110, and 116. RGB values of the color patch 717 may be 169, 23, and 34. RGB values of the color patch 718 may be 120, 19, and 1. RGB values of the color patch 721 may be 176, 62, and 60. RGB values of the color patch 722 may be 77, 142, and 68. RGB values of the color patch 723 may be 42, 76, and 149.

The apparatus 100 for managing a wound may place a red (R) series in the color patches 317 and 721, a green (G) series in the color patches 318 and 722, and a blue (B) series in the color patches 319 and 723. The apparatus 100 for managing a wound may later correct the colors of the wound images using the color patches 317, 318, 319, 721, 722, and 723.

The apparatus 100 for managing a wound may control the color chart 300 to include the first marker 321, the second marker 322, the third marker 323, and the fourth marker 324. The first to fourth markers 321 to 324 may be configured to determine the direction of the color chart 300 and the location of the color included in the color chart 300. The first marker 321 may be located on the upper left of the color chart 300. The second marker 322 may be located on the upper right of the color chart 300. The third marker 323 may be located on the lower left of the color chart 300. The fourth marker 324 may be located on the lower right of the color chart 300. The apparatus 100 for managing a wound may determine the locations of the plurality of color patches using the first to fourth markers 321 to 324.

The color chart 300 may include symbols that the apparatus 100 for managing a wound may recognize. The symbol may have a certain figure. Although not illustrated in FIG. 3, the symbol may include a barcode or a quick response (QR) code. The apparatus 100 for managing a wound may input information on a patient or information on a wound to the apparatus 100 for managing a wound. The apparatus 100 for managing a wound may determine a symbol based on the information on the patient or the information on the wound. Later, the apparatus 100 for managing a wound or the external device may acquire the information on the patient or the information on the wound based on the symbol. The symbols may represent the first to fourth markers.

In addition, the control unit 130 of the apparatus 100 for managing a wound may acquire the area of the color chart 300 based on at least one of the plurality of markers 321, 322, 323, and 324 having different shapes included in the color chart 300. The control unit 130 may include the first marker 321 having a first shape. The control unit 130 may include the fourth marker 324 having a fourth shape. The control unit 130 may include a fourth marker 324 having a fourth shape. The control unit 130 may determine the area of the color chart 300 based on a location of a point based on the first marker 321 and a location of a point based on the fourth marker 324. For example, the location of the point based on the first marker 321 may be an upper left point or a center point of the first marker 321. In addition, a location of a point based on the fourth marker 324 may be a lower right point or a center point of the fourth marker 324.

The shapes of the first to fourth markers 321 to 324 may be different. The first marker 321 may have a first shape, the second marker 322 may have a second shape, the third marker 323 may have a third shape, and the fourth marker 324 may have a fourth shape. The apparatus 100 for managing a wound may later determine an orientation of the color chart 300 based on the first to fourth markers 321 to 324. That is, the apparatus 100 for managing a wound may divide the top, bottom, left, and right of the color chart 300 based on the first to fourth markers 321 to 324. The apparatus 100 for managing a wound may rotate the measured image so that the first marker 321 and the second marker 322 are above the third marker 323 and the fourth marker 324. In addition, the apparatus 100 for managing a wound may process the measured images using the rotated measured images. Therefore, the apparatus 100 for managing a wound does not need to include different algorithms depending on the orientation of the measured image. The apparatus 100 for managing a wound has the advantage of relatively fast operation since it may always use the same algorithm. For example, the colors closest to the first to fourth markers 321 to 324 in the color chart 300 may be different. According to the present disclosure, since the rotating measured image with the same orientation is used, the color patch 311 may always be located at the right of the first marker 321, the color patch 313 may always be located at the left of the second marker 322, the color patch 317 may always be located at the third marker 323, and the color patch 319 may always be located at the left of the fourth marker 324.

For example, the apparatus 100 for managing a wound may acquire the preview image or measured image for the color chart 300. The apparatus 100 for managing a wound may recognize the first marker 321 having the first shape. The apparatus 100 for managing a wound may recognize the second marker 322 having a second shape. The apparatus 100 for managing a wound may locate the first marker 321 on the upper left and the second marker 322 on the upper right. The apparatus 100 for managing a wound may rotate or deform the preview image or the measured image so that the y-axis coordinates of the center point of the first marker 321 and the center point of the second marker 322 are the same.

In addition, the apparatus 100 for managing a wound may recognize the third marker 323 having a third shape. The apparatus 100 for managing a wound may recognize the fourth marker 324 having a fourth shape. The apparatus 100 for managing a wound may locate the third marker 323 on the lower left and the fourth marker 324 on the lower right. The apparatus 100 for managing a wound may rotate or deform the preview image or the measured image so that the y-axis coordinates of the center point of the third marker 323 and the center point of the fourth marker 324 are the same. The apparatus 100 for managing a wound may locate the first marker 321 on the upper left and the third marker 323 on the lower left. The apparatus 100 for managing a wound may rotate or deform the preview image or the measured image so that the x-axis coordinates of the center point of the first marker 321 and the center point of the third marker 323 are the same. The apparatus 100 for managing a wound may locate the second marker 322 on the upper right and the fourth marker 324 on the lower right. The apparatus 100 for managing a wound may rotate or deform the preview image or the measured image so that the x-axis coordinates of the center point of the second marker 322 and the center point of the fourth marker 324 are the same. As described above, since the shapes of the first to fourth markers 321 to 324 are different, the apparatus 100 for managing a wound may automatically determine the up, down, left, and right directions of the measured image or preview image.

In addition, the apparatus 100 for managing a wound may acquire widths of the first to fourth markers 321 to 324 and compare the widths to determine whether the preview image or the measured image are captured at an angle. The width may be calculated using the number of pixels. In addition, the apparatus 100 for managing a wound may correct the preview image or the measured image so that the widths of the first to fourth markers 321 to 324 are the same. Alternatively, the apparatus 100 for managing a wound may acquire the measured image by capturing the color chart 300 when the widths of the first to fourth markers 321 to 324 are the same.

The apparatus 100 for managing a wound may control so that the color chart 300 including the color patches 310 of a plurality of colors determined based on user input and the plurality of markers 321, 322, 323, and 324 is printed. A thickness of the color chart 300 may be 5 mm. Therefore, the color chart may not be easily deformed. In addition, the color chart 300 may be printed on matte-coated paper. Accordingly, the color chart 300 may be less affected by ambient light when captured.

The user may place the printed color chart 300 near the patient's wound and use the image acquisition unit 110 of the apparatus 100 for managing a wound to collect the measured image including the wound image and the image of the color chart 300.

The apparatus 100 for managing a wound may output the information related to the color chart 300 to the display unit. The apparatus 100 for managing a wound may acquire the patient's wound image including the color chart 300 using the image acquisition unit 110. The apparatus 100 for managing a wound may adjust saturation or brightness of the image including the color chart 300 depending on the amount and type of light around the patient. The apparatus 100 for managing a wound may adjust a white balance of the image depending on the amount and type of light around the patient.

The configuration in which the apparatus 100 for managing a wound acquires the image using the image acquisition unit 110 has been described above, but is not limited thereto. The apparatus 100 for managing a wound may acquire the image from the external device using the communication unit. The apparatus 100 for managing a wound may receive the image obtained by capturing the wound along with the color chart 300 from the external device in a wired or wireless manner. The apparatus 100 for managing a wound may acquire the information on the patient or the information on the wound based on the symbol of the color chart 300 included in the image.

The input unit 120 of the apparatus 100 for managing a wound may receive the information on the patient, which includes the sex and age of the patient, from the user. In addition, the input unit 120 of the apparatus 100 for managing a wound may perform an operation of receiving the information on the wound including the wounded area, the wound cause, and the time that the wound has occurred. As described above, the symbol of the color chart 300 may be determined based on the information on the patient and the information on the wound. However, the symbol is not limited thereto, and the apparatus 100 for managing a wound may acquire the information on the patient or the information on the wound based on the symbol of the color chart 300. In addition, the apparatus 100 for managing a wound may not confirm the information on the wound based on the symbol.

The information on the wounded area included in the information on the wound may include at least one of the patient's forehead, eyes, nose, mouth, ear, or cheek. The cause of the wound may include at least one of a burn, a stab, a laceration, or a bite. In addition, the cause of the wound may include detailed causes. For example, when the cause of the wound is a burn, the detailed causes may include at least one of boiling water, flame, electricity, contact, chemical, steam, or friction. The detailed causes may include even more detailed classifications. In addition, the apparatus 100 for managing a wound may display statistical data about the information on the wound on the display unit. The user may confirm the statistical data about the cause of the wound and easily figure out the wound for treatment.

Referring back to FIG. 2, the control unit 130 of apparatus 100 for managing a wound may perform an operation 220 of acquiring the location of the first marker included in the color chart 300 based on the measured image. In addition, the apparatus 100 for managing a wound may perform an operation 230 of acquiring the location of the second marker included in the color chart based on the measured image. In addition, the apparatus 100 for managing a wound may perform an operation 240 of acquiring the location of the third marker included in the color chart based on the measured image. In addition, the apparatus 100 for managing a wound may perform an operation 250 of acquiring the location of the fourth marker included in the color chart based on the measured image. The location of the first marker, the location of the second marker, the location of the third marker, and the location of the fourth marker may be expressed as pixel coordinates of the measured image. The location of the first marker, the location of the second marker, the location of the third marker, and the location of the fourth marker may refer to the coordinates of the pixels (center points) located at the centers of the first to fourth markers. The apparatus 100 for managing a wound may determine the locations of the first to fourth markers 321 to 324 based on a predetermined algorithm. For example, the apparatus 100 for managing a wound may acquire a center point of a marker through an OpenCV library. An upper left corner of the image may have coordinates of (0, 0). In addition, the x-axis coordinate value may increase from left to right. In addition, the y-axis coordinate value may increase from top to bottom.

The apparatus 100 for managing a wound may perform an operation 260 of acquiring the center points of a plurality of color patches 310 included in the color chart based on the location of the first marker, the location of the second marker, the location of the third marker, and the location of the fourth marker. The operation 260 of acquiring the center points of the plurality of color patches 310 will be described with reference to FIG. 4.

FIG. 4 is a diagram for describing the operation of the apparatus for managing a wound according to an embodiment of the present disclosure.

The apparatus 100 for managing a wound may perform the following operations to acquire the center points of the plurality of color patches 310. The apparatus 100 for managing a wound may perform an operation of generating a first horizontal line 420 connecting the location 411 of the first marker 321 and the location 412 of the second marker 322. The apparatus 100 for managing a wound may perform an operation of determining n−1 points by dividing the first horizontal line into n equal parts to be the center points of the plurality of color patches. Here, n may be a predetermined natural number. For example, referring to FIG. 4, n may be 4. The apparatus 100 for managing a wound may perform an operation of determining a first point 421, a second point 422, and a third point 423 by quartering the first horizontal line 420 to be the center points of the plurality of color patches. That is, the first horizontal line 420 may be quartered by the first point 421, the second point 422, and the third point 423. The first point 421 may be the center point of the first color patch 311. The second point 422 may be the center point of the second color patch 312. The third point 423 may be the center point of the third color patch 313.

Referring to FIG. 7, n may be 5. The apparatus 100 for managing a wound may perform an operation of determining 4 points by dividing the first horizontal line into five equal parts to be the center points of the plurality of color patches. The method of determining the center points of the plurality of color patches is omitted because it overlaps with FIG. 3.

In addition, the apparatus 100 for managing a wound may perform the following operations to acquire the center points of the plurality of color patches. The apparatus 100 for managing a wound may perform an operation of generating a first vertical line 430 connecting the location 411 of the first marker 321 and the location 413 of the third marker 323. In addition, the apparatus 100 for managing a wound may perform an operation of generating a second vertical line 433 connecting the location 412 of the second marker 322 and the location 414 of the fourth marker 324.

The apparatus 100 for managing a wound may perform an operation of bisecting the first vertical line 430 to determine the first sub point 431. That is, the first sub point 431 may be a point bisecting the first vertical line 430. In addition, the apparatus 100 for managing a wound may perform an operation of bisecting the second vertical line 433 to determine a second sub point 432. That is, the second sub point 432 may be a point bisecting the second vertical line 433. However, the second sub point 432 is not limited thereto. The apparatus 100 for managing a wound may acquire a plurality of left sub points by dividing the first vertical line 430 into h equal parts. In addition, the apparatus 100 for managing a wound may acquire a plurality of right sub points by dividing the second vertical line 433 into h equal parts.

The apparatus 100 for managing a wound may perform an operation of generating a second horizontal line 440 connecting the first sub point 431 and the second sub point 432. The apparatus 100 for managing a wound may perform an operation of determining m−1 points by dividing the second horizontal line into m equal parts to be the center points of the plurality of color patches. Here, m may be a predetermined natural number. In addition, m may be equal to n. For example, referring to FIG. 4, m may be 4. The apparatus 100 for managing a wound may perform an operation of determining a fourth point 441, a fifth point 442, and a sixth point 443 by quartering the second horizontal line 440 to be the center points of the plurality of color patches. That is, the second horizontal line 440 may be quartered by the fourth point 441, the fifth point 442, and the sixth point 443. The fourth point 441 may be the center point of the fourth color patch 314. The fifth point 442 may be the center point of the fifth color patch 315. The sixth point 443 may be the center point of the sixth color patch 316.

Referring to FIG. 7, m may be 5. The apparatus 100 for managing a wound may perform an operation of determining 4 points by dividing the second horizontal line into five equal parts to be the center points of the plurality of color patches. The method of determining the center points of the plurality of color patches is omitted because it overlaps with FIG. 3.

In addition, the apparatus 100 for managing a wound may perform the following operations to acquire the center points of the plurality of color patches. The apparatus 100 for managing a wound may perform an operation of generating a third horizontal line 450 connecting the location 413 of the third marker 323 and the location 414 of the fourth marker 324.

The apparatus 100 for managing a wound may perform an operation of determining k−1 points by dividing the third horizontal line into k equal parts to be the center points of the plurality of color patches. Here, k may be a predetermined natural number. In addition, k may be different from n. However, it is not limited thereto and k may be equal to n. For example, referring to FIG. 4, k may be 4. The apparatus 100 for managing a wound may perform an operation of determining a seventh point 451, an eighth point 452, and a ninth point 453 by quartering the third horizontal line 450 to be the center points of the plurality of color patches. That is, the third horizontal line 450 may be quartered by the seventh point 451, the eighth point 452, and the ninth point 453. The seventh point 451 may be the center point of the seventh color patch 317. The eighth point 452 may be the center point of the eighth color patch 318. The ninth point 453 may be the center point of the ninth color patch 319. The first to ninth points 421 to 453 may be expressed as pixel coordinate values of the measured image.

The apparatus 100 for managing a wound may acquire the actual distance per pixel of the measured image. The actual distance is a physical distance and may have units of mm, cm, or m. The apparatus 100 for managing a wound may determine how far the distance between two pixels in an image is in reality. After acquiring the actual distance per pixel, the apparatus 100 for managing a wound may determine how far the distance between two pixels in an image is in reality.

The apparatus 100 for managing a wound may perform an operation of acquiring a first pixel length by dividing the pixel length of the first horizontal line into n equal parts. Referring to FIG. 3, the apparatus 100 for managing a wound may perform an operation of acquiring the first pixel length by quartering the pixel length of the first horizontal line 420. The pixel length of the first horizontal line 420 may be a value obtained by subtracting the x-axis value of the location 411 of the first marker 321 from the x-axis value of the location 412 of the second marker 322. The apparatus 100 for managing a wound may acquire ¼ of the pixel length of the first horizontal line 420 as the first pixel length. Referring to FIG. 7, the apparatus 100 for managing a wound may perform an operation of acquiring the first pixel length by dividing the pixel length of the first horizontal line 420 into five equal parts. The pixel length of the first horizontal line 420 may be a value obtained by subtracting the x-axis value of the location 411 of the first marker 321 from the x-axis value of the location 412 of the second marker 322. The apparatus 100 for managing a wound may acquire ⅕ of the pixel length of the first horizontal line 420 as the first pixel length.

The apparatus 100 for managing a wound may perform an operation of acquiring a second pixel length by dividing the pixel length of the second horizontal line into m equal parts. Referring to FIG. 3, the apparatus 100 for managing a wound may perform an operation of acquiring the second pixel length by quartering the pixel length of the second horizontal line 440. The pixel length of the second horizontal line 440 may be a value obtained by subtracting the x-axis value of the first sub point 432 from the x-axis value of the second sub point 431. The apparatus 100 for managing a wound may acquire ¼ of the pixel length of the second horizontal line 440 as the second pixel length. Referring to FIG. 7, the apparatus 100 for managing a wound may acquire ⅕ of the pixel length of the second horizontal line 440 as the second pixel length.

The apparatus 100 for managing a wound may perform an operation of acquiring a third pixel length by dividing the pixel length of the third horizontal line into k equal parts. Referring to FIG. 3, the apparatus 100 for managing a wound may perform an operation of acquiring the third pixel length by quartering the pixel length of the third horizontal line 450. The pixel length of the third horizontal line 450 may be a value obtained by subtracting the x-axis value of the location 413 of the third marker 323 from the x-axis value of the location 414 of the fourth marker 324. The apparatus 100 for managing a wound may acquire the third pixel length by quartering the pixel length of the third horizontal line 450. Referring to FIG. 7, the apparatus 100 for managing a wound may acquire ¼ of the pixel length of the third horizontal line 450 as the third pixel length.

The apparatus 100 for managing a wound may perform an operation of acquiring a fourth pixel length by dividing the pixel length of the first vertical line 430 into h equal parts. Referring to FIGS. 3 and 7, the apparatus 100 for managing a wound may perform an operation of acquiring the fourth pixel length by bisecting the pixel length of the first vertical line 430. The pixel length of the first vertical line 430 may be a value obtained by subtracting the y-axis value of the location 411 of the first marker 321 from the y-axis value of the location 413 of the third marker 323. The apparatus 100 for managing a wound may acquire the fourth pixel length by bisecting the pixel length of the first vertical line 430.

The apparatus 100 for managing a wound may perform an operation of acquiring a fifth pixel length by dividing the pixel length of the second vertical line 433 into h equal parts. Referring to FIGS. 3 and 7, the apparatus 100 for managing a wound may perform an operation of acquiring the fifth pixel length by bisecting the pixel length of the second vertical line 433. The pixel length of the second vertical line 433 may be a value obtained by subtracting the y-axis value of the location 412 of the second marker 322 from the y-axis value of the location 414 of the fourth marker 324. The apparatus 100 for managing a wound may acquire the fifth pixel by bisecting the pixel length of the second vertical line 433.

The apparatus 100 for managing a wound may perform an operation of determining a representative pixel length using at least one of the first to fifth pixel lengths. For example, the apparatus 100 for managing a wound may determine an average value or a median value of the first to fifth pixel lengths to be the representative pixel length. In addition, the apparatus 100 for managing a wound may perform an operation of determining a representative pixel length using at least one of the first pixel length, the second pixel length, and the third pixel length. For example, the apparatus 100 for managing a wound may determine an average value or a median value of the first to third pixel lengths to be the representative pixel length.

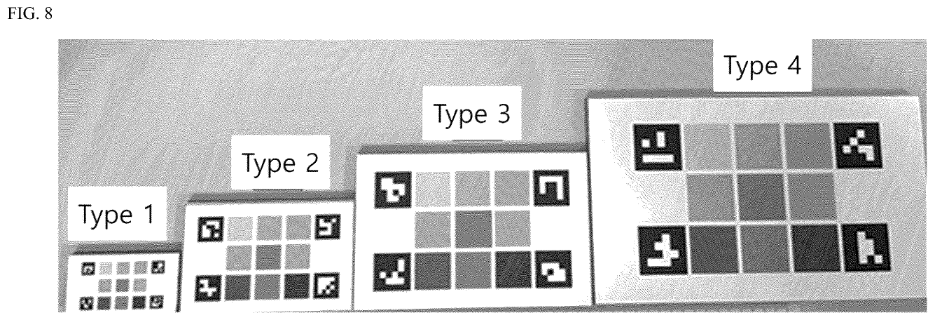

The apparatus 100 for managing a wound may perform an operation of acquiring a predetermined actual distance between the center points of the plurality of color patches. The predetermined actual distance between the center points of the plurality of color patches may be the predetermined actual distance between the center points of two color patches. This will be described with reference to FIGS. 5 and 8.

FIG. 5 is a table for describing a predetermined actual distance between center points of color patches according to an embodiment of the present disclosure. FIG. 8 is a diagram illustrating color charts of a plurality of sizes according to an embodiment of the present disclosure.

According to an embodiment of the present disclosure, as illustrated in FIG. 8, the color chart 300 may have four types. Each of the four types may include color patches of different sizes. In one color chart 300, the size of the color patch may be the same as the size of the marker. Each of the plurality of color patches 310 included in the color chart 300 may be a square.

Referring to FIG. 8, the shape of the marker may be different for each type. Therefore, the apparatus 100 for managing a wound may determine actual one-side lengths of the plurality of color patches 310 of the measured image based on the marker. The apparatus 100 for managing a wound may have pre-stored a table as illustrated in FIG. 5. The apparatus 100 for managing a wound may recognize the shape of the marker when a color patch such as type 1 in FIG. 8 is captured, and determine that the one-side length of each of the plurality of color patches 310 is 5 mm and the distance between the plurality of color patches is 2 mm. The apparatus 100 for managing a wound may recognize the shape of the marker when a color patch such as type 2 in FIG. 8 is captured, and determine that the one-side length of each of the plurality of color patches 310 is 10 mm and the distance between the plurality of color patches is 2 mm. The apparatus 100 for managing a wound may recognize the shape of the marker when a color patch such as type 3 in FIG. 8 is captured, and determine that the one-side length of each of the plurality of color patches 310 is 15 mm and the distance between the plurality of color patches is 2 mm. The apparatus 100 for managing a wound may recognize the shape of the marker when a color patch such as type 4 in FIG. 8 is captured, and determine that the one-side length of each of the plurality of color patches 310 is 20 mm and the distance between the plurality of color patches is 2 mm.

In addition, the apparatus 100 for managing a wound may determine whether the color chart 300 included in the measured image is normal based on the first to fourth markers. The apparatus 100 for managing a wound may determine whether the first marker is in the upper left. When the first marker is not in the upper left, the measured image may rotate so that the first marker is in the upper left. Next, the apparatus 100 for managing a wound may confirm whether the second marker is in the upper right. When the second marker is not in the upper right, the apparatus 100 for managing a wound may determine that the color chart 300 included in the measured image is abnormal. The apparatus 100 for managing a wound may check whether the third marker is in the lower left. When the third marker is not in the lower left, the apparatus 100 for managing a wound may determine that the color chart 300 included in the measured image is abnormal. The apparatus 100 for managing a wound may check whether the fourth marker is in the lower right. When the fourth marker is not in the lower right, the apparatus 100 for managing a wound may determine that the color chart 300 included in the measured image is abnormal.

In addition, the apparatus 100 for managing a wound may determine the location of the first marker. The apparatus 100 for managing a wound may determine the location of the second marker. The apparatus 100 for managing a wound may determine the location of the third marker. The apparatus 100 for managing a wound may rotate the measured image so that the location of the second marker is located at the right of the first marker and the third marker is located at the lower end of the first marker. When it is impossible for the apparatus 100 for managing a wound to rotate the measured image so that the location of the second marker is located at the right of the first marker and the third marker is located at the lower end of the first marker, the apparatus 100 for managing a wound may determine that the color chart 300 included in the measured image is abnormal.

In this way, the apparatus 100 for managing a wound may determine that the color chart 300 is abnormal, thereby preventing anyone from receiving services by the apparatus 100 for managing a wound. Therefore, the security of the apparatus 100 for managing a wound may increase.

Each of the plurality of color patches 310 may be one of squares whose one-side lengths are 5 mm, 10 mm, 15 mm, and 20 mm. The distance between the plurality of color patches may be 2 mm. As illustrated in FIG. 3, the distances between the closest color patches and the markers in one color patch may all be constant. That is, the distances between the horizontally closest color patches and the markers in one color patch may all be 2 mm. As illustrated in FIG. 7, the distances between the closest color patches and the markers in one color patch may not be constant. For example, an interval between patches of two rows on the top may be 2 mm. However, the interval between patches in the last row may be 2 mm or more.

In this way, since the one-side lengths of the plurality of color patches 310 are determined and the distance between the plurality of color patches 310 is determined, the apparatus 100 for managing a wound may acquire a predetermined actual distance between the center points of two color patches. For example, in the case of type 1, the predetermined actual distance between the center points of two color patches may be 7 mm (=5 mm+2 mm). In the case of type 2, the predetermined actual distance between the center points of two color patches may be 12 mm (=10 mm+2 mm). In the case of type 3, the predetermined actual distance between the center points of two color patches may be 17 mm (=15 mm+2 mm). In the case of type 4, the predetermined actual distance between the center points of two color patches may be 22 mm (=20 mm+2 mm).

The apparatus 100 for managing a wound may perform an operation of acquiring the actual distance per pixel using the predetermined actual distance and the representative pixel length. More specifically, the apparatus for managing a wound may divide the predetermined actual distance by the representative pixel length. For example, in type 1, when the representative pixel length is 25 pixels, the apparatus 100 for managing a wound may determine the actual distance per pixel as 7/25 (mm/pixel). The apparatus 100 for managing a wound may measure the actual length of the patient's wound shown in the measured image using the actual distance per pixel.

For reference, in the case of type 1, a distance between an upper line of a marker or a patch in a first row and a lower line of a marker or a hatch in of a last row may be 19 mm. A distance between a left line of a marker or a patch in a left column and a right line of a marker or a hatch in a right column may be 33 mm. In the case of type 2, a distance between the upper line of the marker or patch in the first row and the lower line of the marker or hatch in the last row may be 34 mm. The distance between the left line of the marker or patch in the left column and the right line of the marker or hatch in the right column may be 58 mm. In the case of type 3, the distance between the upper line of the marker or patch in the first row and the lower line of the marker or hatch in the last row may be 49 mm. The distance between the left line of the marker or patch in the left column and the right line of the marker or hatch in the right column may be 83 mm. In the case of type 4, the distance between the upper line of the marker or patch in the first row and the lower line of the marker or hatch in the last row may be 64 mm. The distance between the left line of the marker or patch in the left column and the right line of the marker or hatch in the right column may be 108 mm.

The apparatus 100 for managing a wound may perform an operation of acquiring the center points of the plurality of color patches 310 and then correcting the red color of the measured image based on the pixel value of the seventh point 451. As described above, the seventh point 451 may be the center point of the seventh color patch 317. The seventh color patch 317 may have a red color. The seventh color patch 317 may actually have a pre-designated red color code. However, depending on the capturing environment, the seventh point 451 in the measured image may not be the pre-designated red color code. The apparatus 100 for managing a wound may correct the measured image so that the pixel value at the seventh point 451 has the pre-designated red color code.

In addition, the apparatus 100 for managing a wound may correct the red color of the measured image based on the pixel value around the seventh point. The area around the seventh point may refer to pixels included within a predetermined distance from the seventh point 451. The apparatus 100 for managing a wound may correct the measured image so that the average value of pixel values around the seventh point has a designated red color.

The apparatus 100 for managing a wound may perform an operation of correcting the green color of the measured image based on the pixel value of the eighth point 452. As described above, the eighth point 452 may be the center point of the eighth color patch 318. The eighth color patch 318 may have a green color. The eighth color patch 318 may actually have a pre-designated green color code. However, depending on the capturing environment, the eighth point 452 in the measured image may not be the pre-designated green color code. The apparatus 100 for managing a wound may correct the measured image so that the pixel value at the eighth point 452 has the pre-designated green color code.

In addition, the apparatus 100 for managing a wound may correct the green color of the measured image based on the pixel value around the eighth point. The area around the eighth point may refer to pixels included within a predetermined distance from the eighth point 452. The apparatus 100 for managing a wound may correct the measured image so that the average value of pixel values around the eighth point has a designated green color.

The apparatus 100 for managing a wound may perform an operation of correcting the blue color of the measured image based on the pixel value of the ninth point 453. As described above, the ninth point 453 may be the center point of the ninth color patch 319. The ninth color patch 319 may have a blue color. The ninth color patch 319 may actually have the pre-designated blue color code. However, depending on the capturing environment, the ninth point 453 in the measured image may not be the pre-designated blue color code. The apparatus 100 for managing a wound may correct the measured image so that the pixel value at the ninth point 453 has the pre-designated blue color code.

In addition, the apparatus 100 for managing a wound may correct the blue color of the measured image based on the pixel value around the ninth point. The area around the ninth point may refer to pixels included within a predetermined distance from the ninth point 453. The apparatus 100 for managing a wound may correct the measured image so that the average value of pixel values around the ninth point has a designated blue color.

The apparatus 100 for managing a wound may perform an operation of deriving the wounded area from the measured image. The control unit 130 may determine whether a specific area in the acquired image is included in the wounded area. The apparatus 100 for managing a wound may derive the wounded area based on a difference between a normal skin and a wounded skin appearing in the image. The image of the normal skin may include a uniform texture and pattern. However, the wounded area may not have a uniform texture and pattern.

The control unit 130 of the apparatus 100 for managing a wound may perform an operation of deriving the area of the color chart 300 from the image. The apparatus 100 for managing a wound may derive the area of the color chart based on a plurality of colors being arranged at regular intervals. The apparatus 100 for managing a wound may derive the area of the color chart based on a symbol made of dots or lines representing the area of the color chart. The apparatus 100 for managing a wound may derive a color chart area based on the texture, shape, or color of a color chart different from that of the skin.

The apparatus 100 for managing a wound may perform an operation of acquiring color analysis results by comparing at least two different colors included in the color chart area and the color of the wounded area. The apparatus 100 for managing a wound may express the color analysis results as a ratio of the number of pixels of different colors included in the wounded area.

More specifically, the control unit of the apparatus 100 for managing a wound may acquire the ratio of the a-th number of pixels in the wounded area having the same color as A-th color included in one of the first color patch 311 to the sixth color patch 316 of the color chart 300 and the number of pixels included in the wounded area as an x-th color analysis result. Here, a and x may be natural numbers. In addition, the control unit of the apparatus 100 for managing a wound may acquire the ratio of the a-th number of pixels in the wounded area having the same color as the A-th color included in the color chart area and the b-th number of pixels in the wounded area having the same color as a B-th color included in one of the color chart area and the first color patch 311 to the sixth color patch 316 of the color chart 300 as a y-th color analysis result. Here a, b and y may be natural numbers.

According to an embodiment of the present disclosure, the control unit 130 of the apparatus 100 for managing a wound may acquire the ratio of the first number of pixels in the wounded area having the same color as a first color included in one of the first color patch 311 to the sixth color patch 316 of the color chart and the entire number of pixels included in the wounded area as a first color analysis result. In addition, the control unit 130 of the apparatus 100 for managing a wound may acquire the ratio of the second number of pixels in the wounded area having the same color as a second color included in one of the first color patch 311 to the sixth color patch 316 of the color chart and the entire number of pixels included in the wounded area as a second color analysis result. In addition, the control unit 130 of the apparatus 100 for managing a wound may obtain the ratio of the first number and the second number as a third color analysis result. The apparatus 100 for managing a wound may display the first color analysis result, the second color analysis result, or the third color analysis result as real numbers.

The apparatus 100 for managing a wound may output the first color analysis result, the second color analysis result, or the third color analysis result to the output unit. For convenience of description, the description is made based on two colors, such as the first color and the second color, but the apparatus 100 for managing a wound may derive the color analysis results based on three or more colors.

According to another embodiment of the present disclosure, the control unit 130 of the apparatus 100 for managing a wound may acquire a ratio of the number obtained by subtracting the third number of pixels included in the wounded area having a color not included in the color chart from the total number of pixels included in the wounded area and the first number as the first color analysis result. Here, the first number may be the number of pixels in the wounded area having the same color as the first color included in the color chart area.

In addition, the control unit 130 of the apparatus 100 for managing a wound may acquire the ratio of the number obtained by subtracting the third number from the total number of pixels included in the wounded area and the second number as the second color analysis result. Here, the second number may be the number of pixels in the wounded area having the same color as the second color included in the color chart area.

According to an embodiment of the present disclosure, the control unit 130 of apparatus 100 for managing a wound may acquire the information on the plurality of locations of pixels in the wounded area having the same color as the color as the A-th color included in the color chart area. Alternatively, the control unit 130 may acquire information on the shape of pixels in the wounded area having the same color as the A-th color included in the color chart area as the analysis result.

The control unit 130 of the apparatus 100 for managing a wound may perform an operation of acquiring the size of the wound based on the actual distance per pixel. The process of acquiring the actual distance per pixel has already been described, and therefore, detailed description thereof will be omitted. The apparatus 100 for managing a wound may measure the left and right pixel distances at the left and right ends of the wounded area. In addition, the apparatus 100 for managing a wound may measure the upper and lower pixel distances of the upper and lower ends of the wounded area. In addition, the apparatus 100 for managing a wound may acquire the left and right actual distances and the upper and lower actual distances of the wound by multiplying the left and right pixel distances and the upper and lower pixel distances by the actual distance per pixel. The apparatus 100 for managing a wound may determine the left and right actual distances and the upper and lower actual distances of the wound to be the size of the wound.

The control unit 130 may perform an operation of searching for the most similar image among the wound images pre-stored in a database based on the information on the patient, the information on the wound, the color analysis results, and the size of the wound.

The database may be stored in the memory 140 of the apparatus 100 for managing a wound, but is not limited thereto. The control unit 130 may transmit and receive data to and from a remote database through the communication unit.

The control unit 130 may determine the similarities of the information on the patient, the information on the wound, the color analysis results, and the size of the wound between the current patient and the patient recorded in the database, respectively. In addition, the control unit 130 may assign a weight to each similarity. The control unit 130 may determine a final similarity based on the similarity and weight. The control unit 130 may acquire the patient's wound image recorded in the database with the highest similarity.

The control unit 130 may select an image having the color analysis result most similar to at least one of the first color analysis result, the second color analysis result, and the third color analysis result as the most similar image.

The control unit 130 may determine the similarity between the color analysis results of the current patient's wound and the patient's wound recorded in the database. The control unit 130 may determine the similarity by comparing the first color analysis result, the second color separation result, or the third color analysis result as described above. The first to third color analysis results may have priorities. For example, when the current patient's wound matches the third color analysis result of multiple patients recorded in the database, the control unit 130 may further compare the first color analysis result and the second color analysis result. The control unit 130 may acquire the patient's wound image recorded in the database that is most similar to the current patient's wound.

The control unit 130 may compare the pattern or shape formed by one color in the image in addition to the first to third color analysis results. For example, when the first color forms a donut shape and the second color forms a circular pattern located in the center of the donut formed by the first color, the control unit 130 may acquire an image having a similar pattern among the patient's wound images stored in the database.

The control unit 130 may perform an operation 270 of acquiring treatment information corresponding to the most similar image. The control unit 130 may acquire the patient's wound image recorded in the database that is most similar to the current patient's wound. The database may store not only the patient's wound image but also the treatment information corresponding to the patient's wound image. The treatment information may represent a record of the treatment process of the wound.

The treatment information may include at least one of the treatment methods corresponding to at least two images showing the change in the wound over time or the image most similar to the current patient's wound. In addition, the treatment information may include at least one of pieces of information on a wound treatment method, an image captured during the wound treatment process, a hospital where treatment was performed, a doctor who treats a wound, medicines used for treatment, and tools used for treatment.

The control unit 130 may perform an operation of outputting the acquired treatment information. The acquired treatment information may be output from the output unit. The user may know a method of treating a wound or whether a visit to a hospital is essential based on the acquired treatment information.

The control unit 130 may perform an operation of determining whether the visit to the hospital is essential based on the treatment information. The control unit 130 may determine the need to visit the hospital when the acquired size of the wound is greater than or equal to a threshold value. The acquired size of the wound may be the width of the wound. In addition, the acquired size of the wound may be the diameter and maximum length of the wound.

As described above, the control unit 130 may acquire an image most similar to the current patient's wound image from the database. The database may store the most similar images together with the treatment information corresponding to the most similar images. The treatment information stored in the database may include the existing method of treating a patient wound and the treatment progress. The control unit 130 may determine whether the visit to the hospital is essential among the treatment methods included in the treatment information. For example, the control unit 130 may determine the need to visit the hospital when the essential drug used in the treatment method is a drug that requires a prescription. In addition, the control unit 130 may determine the need to visit the hospital when surgery is included among the treatment methods. The control unit 130 may output the need to visit the hospital through the output unit. In addition, the control unit 130 may output the reason for the need to visit the hospital through the output unit. The user may easily know the need to visit the hospital and the reason for the need to visit the hospital is necessary based on the contents displayed on the output unit.

The control unit 130 may acquire hospital information when it determines that the visit to the hospital is essential.

Specifically, when the visit to the hospital is essential, the control unit 130 may perform an operation of requesting the hospital information from the server. A server may include a processor and a memory. The server may store a database including the information on the hospital and the treatment information.

The server may perform an operation of transmitting the hospital information to the apparatus 100 for managing a wound. The apparatus 100 for managing a wound may perform an operation of outputting the hospital information through the output unit.

The apparatus 100 for managing a wound may acquire the hospital information stored in the memory of the apparatus 100 for managing a wound. In addition, the apparatus 100 for managing a wound may output the acquired hospital information through the output unit. In this case, the apparatus 100 for managing a wound may not require the server for the hospital information.

The hospital information may include the information on the hospital that provides the image most similar to the image of the current patient's wound acquired by the control unit 130. In addition, the hospital information may include at least one of the time taken for wound treatment by the hospital that provides the most similar image, the amount charged for wound treatment, or the number of times the patient was requested to visit the hospital.

A patient may know information on a hospital that treats a wound most similar to his/her own wound. The more the information on the wound treatment the hospital provides, the more likely the hospital will be exposed to its services. In this way, service providers may encourage a hospital to voluntarily provide treatment information for marketing purposes.

The hospital information may include at least one of descriptions of hospital contact information, a location of a hospital, a distance between a hospital and a patient, transportation to a hospital, hospital business hours, information on medical personnel affiliated with a hospital, an opening date of a hospital, the number of times of reuse by patients at a hospital, a hospital rating, a current waiting list at a hospital, a parking space near a hospital, and hospital facilities.

A user may easily know information on a hospital for wound treatment. In addition, a patient may easily know a hospital that has experience treating his/her wound. In addition, a patient may easily know which hospital treats his/her wound the fastest. In addition, a patient may easily know which hospital treats his/her wound at the lowest cost. In addition, a patient may easily know which hospital treated his/her wound with fewer visits to a hospital.

The control unit 130 may perform an operation of receiving reservation information related to a hospital reservation from the user through the input unit 120. The reservation information may include at least one of a hospital a patient is scheduled to visit, a time when a patient is scheduled to visit, and information on a patient's preferred healthcare provider.

The control unit 130 may perform an operation 522 of transmitting the information on the patient and the reservation information to the hospital server. The server may perform an operation of determining whether a reservation is available. When it is determined that the reservation is possible, the server may perform an operation of transmitting a reservation confirmation signal to the apparatus 100 for managing a wound.

The apparatus 100 for managing a wound may search patient's insurance subscription details. In addition, when the patient's insurance is related to the patient's wound, the apparatus 100 for managing a wound may present the insurance related to the wound to the patient. The patient may reduce costs related to treatment based on their insurance. When the patient does not subscribe the insurance related to the patient's wound, the apparatus 100 for managing a wound may suggest the insurance related to the wound.

The apparatus 100 for managing a wound may display wound-related statistics on the display unit based on medical information stored in the database. The apparatus 100 for managing a wound may store statistical data for each part and factor of a burn. In addition, the apparatus 100 for managing a wound may store monthly burn statistics. The apparatus 100 for managing a wound may display the statistical data on the display unit based on the user's request.

For example, the database of the apparatus 100 for managing a wound may store an age, a sex, an occupation, and a place of residence of a patient and the monthly number of cases of boiling burn, flame burn, electrical burn, contact burns, low-temperature burn, chemical burn, vapor burn, friction burn, sunlight burn, and inhalation burn. The apparatus 100 for managing a wound may confirm the trend of the cause of the burn based on the user's input. The user may easily know how to watch out for burns by looking at the trend in the causes of burns.

The user may easily know the cause of the wound that require monthly attention based on the information provided by the apparatus 100 for managing a wound. Therefore, the user may be careful to avoid wounds.

The apparatus 100 for managing a wound may increase an insurance subscription rate by presenting the main cause of the wound and the insurance related to the wound.

In addition, according to the apparatus 100 for managing a wound according to an embodiment of the present disclosure, the user may roughly determine the patient's conditions without visiting a medical professional, thereby increasing convenience and preventing the patient from spending excessive costs on the wound treatment.

In addition, according to an embodiment of the present disclosure, a medical professional may improve the accuracy of wound diagnosis by making a diagnosis with reference to the apparatus 100 for managing a wound.

In addition, since the apparatus 100 for managing a wound of the present disclosure judges wounds based on the same criteria, the possibility of receiving different judgments depending on the medical professional may be reduced. For example, in the case of second-degree burns, the insurance coverage may vary depending on whether the burn is superficial or deep, and medical professionals may make different judgments. Therefore, an insurance company and a patient may sometimes dispute on whether the burn is deep or superficial. However, since the apparatus 100 for managing a wound of the present disclosure is judged based on the same standard, the dispute between the insurance company and the patient may be reduced.

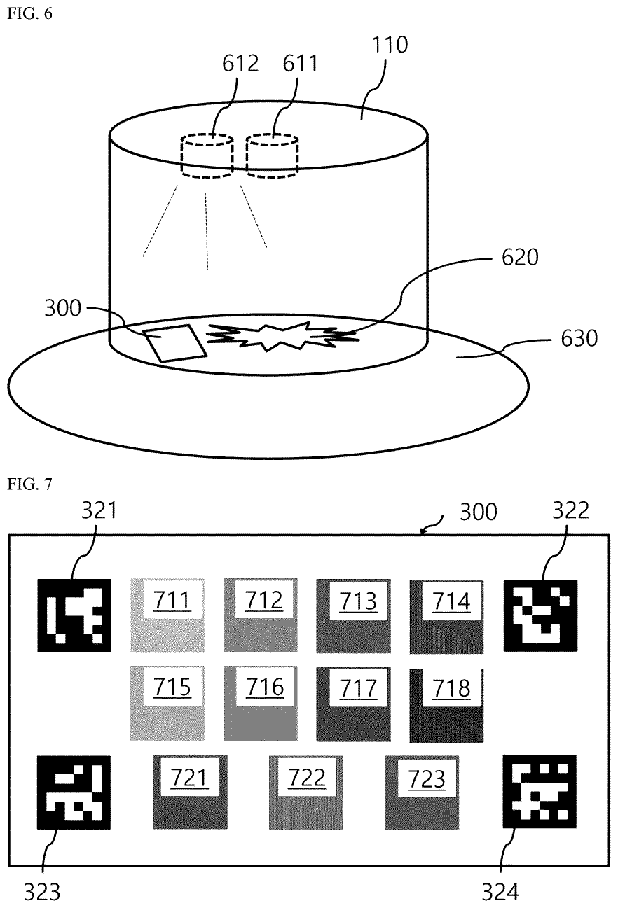

FIG. 6 is a diagram for describing an image acquisition unit according to an embodiment of the present disclosure.

The image acquisition unit 110 may be cylindrical whose lower surface is open. A camera 611 and a light source 612 may be formed on the upper surface of the image acquisition unit 110. When a user places the image acquisition unit 110 on the user's skin surface 630, the inside of the image acquisition unit 110 may be very dark because the lower surface is blocked. The inner surface of the cylindrical image acquisition unit 110 may be white. The inner surface of the cylindrical image acquisition unit 110 may be painted with white matte paint. Therefore, light from the light source may be diffusely reflected. Therefore, the inside of the image acquisition unit 110 may be illuminated by the light source 612. The user may place the color chart 300 around the wound and cover the color chart 300 with the image acquisition unit 110 to acquire the measured image including the wound and the color chart 300. The apparatus 100 for managing a wound may control light around the wound 620 and the color chart 300 using the light source 612 when acquiring the measured image. Therefore, the apparatus 100 for managing a wound may obtain the measured image in a uniform state.

The light source 612 may include a red light source, a blue light source, and a green light source. In addition, the light source 612 may include a white light source. The light source 612 may include a diffusion plate (scattering plate). That is, the light from the light source 612 may be scattered by the scattering plate and radiated into the inside of the image acquisition unit. Therefore, the light source 612 does not radiate onto a specific area but may evenly illuminate the inside of the cylindrical image acquisition unit 110. The light source 612 may illuminate the inside of the image acquisition unit 110 with 20 lux or more. For example, the light source 612 may illuminate the inside of the image acquisition unit 110 with 100 lux. The apparatus 100 for managing a wound may acquire the measured image by performing the operation 210 while light is controlled using the light source 612. Therefore, the analysis results of the apparatus for managing a wound may be more accurate.

So far, various embodiments have been mainly described. It will be understood by those skilled in the art to which the present disclosure pertains that the present disclosure may be implemented in a modified form without departing from essential characteristics of the present disclosure. Therefore, the embodiments disclosed herein should be considered in an illustrative aspect rather than a restrictive aspect. The scope of the present disclosure should be defined by the claims rather than the above description, and equivalents to the claims should be interpreted to fall within the present disclosure.

Meanwhile, the above-described embodiments of the present disclosure can be written as a program that may be executed on a computer, and can be implemented in a general-purpose digital computer that operates the program using a computer-readable recording medium. The computer-readable recording medium includes storage media such as magnetic storage media (e.g., a ROM, a floppy disk, a hard disk, etc.) and optically readable media (e.g., a compact disc read-only memory (CD-ROM), a digital video disc (DVD), etc.).

1. A method of operating an apparatus for managing a wound that captures a color chart, comprising:
acquiring a measured image including the color chart;
acquiring a location of a first marker included in the color chart based on the measured image;
acquiring a location of a second marker included in the color chart based on the measured image;
acquiring a location of a third marker included in the color chart based on the measured image;
acquiring a location of a fourth marker included in the color chart based on the measured image; and
acquiring center points of a plurality of color patches included in the color chart based on the location of the first marker, the location of the second marker, the location of the third marker, and the location of the fourth marker.

2. The method of claim 1, wherein the first marker is located at an upper left of the color chart, the second marker is located at an upper right of the color chart, the third marker is located at a lower left of the color chart, and the fourth marker is located at a lower right of the color chart.

3. The method of claim 2, wherein the acquiring of the center points of the plurality of color patches includes:
generating a first horizontal line connecting the location of the first marker and the location of the second marker; and
determining n−1 points dividing the first horizontal line into n equal parts to be the center points of the plurality of color patches.

4. The method of claim 3, wherein the acquiring of the center points of the plurality of color patches includes:
generating a first vertical line connecting the location of the first marker and the location of the third marker;
generating a second vertical line connecting the location of the second marker and the location of the fourth marker;
determining a first sub point by bisecting the first vertical line;
determining a second sub point by bisecting the second vertical line;
generating a second horizontal line connecting the first sub point and the second sub point; and
determining m−1 points by dividing the second horizontal line into m equal parts to be the center points of the plurality of color patches.

5. The method of claim 4, wherein the acquiring of the center points of the plurality of color patches includes:
generating a third vertical line connecting the location of the third marker and the location of the fourth marker; and
determining k−1 points by dividing a third horizontal line into k equal parts to be center points of the plurality of color patches.

6. The method of claim 5, further comprising:
acquiring a first pixel length by dividing a pixel length of the first horizontal line into n equal parts;
acquiring a second pixel length by dividing a pixel length of the second horizontal line into m equal parts;
acquiring a third pixel length by dividing a pixel length of the third horizontal line into k equal parts;
determining a representative pixel length using at least one of the first pixel length, the second pixel length, and the third pixel length;
acquiring a predetermined actual distance between the center points of the plurality of color patches; and
acquiring an actual distance per pixel using the predetermined actual distance and the representative pixel length.

7. The method of claim 6, further comprising:
determining a seventh point, an eighth point, and a ninth point by quartering the third horizontal line to be center points of the plurality of color patches;
acquiring the center points of the plurality of color patches, and then correcting a red color of the measured image based on a pixel value of the seventh point;
correcting a green color of the measured image based on a pixel value of the eighth point; and
correcting a blue color of the measured image based on a pixel value of the ninth point.

8. The method of claim 7, wherein each of the plurality of color patches included in the color chart is one of squares whose one-side lengths are 5 mm, 10 mm, 15 mm, and 20 mm, and a distance between the plurality of color patches is 2 mm.

9. The method of claim 1, wherein the acquiring of the measured image including the color chart includes:
acquiring a preview image including the color chart;
acquiring the location of the first marker included in the color chart based on the preview image;
acquiring the location of the second marker included in the color chart based on the preview image;
acquiring the location of the third marker included in the color chart based on the preview image;
acquiring the location of the fourth marker included in the color chart based on the preview image;
determining whether a first difference value between a y-axis value of the location of the first marker and a y-axis value of the location of the second marker is within a critical distance;
determining whether a second difference value between a y-axis value of the location of the third marker and a y-axis value of the location of the fourth marker is within the critical distance;
determining whether a third difference value between an x-axis value of the location of the first marker and an x-axis value of the location of the third marker is within the critical distance;
determining whether a fourth difference value between an x-axis value of the location of the second marker and an x-axis value of the location of the fourth marker is within the critical distance; and
when the first difference value, the second difference value, the third difference value, and the fourth difference value are all within the critical distance, acquiring the preview image as the measured image.

10. The method of claim 9, further comprising:
displaying a predetermined icon at the location of the first marker on the preview image;
displaying a fourth horizontal line on the preview image, which is parallel to a horizontal axis of the preview image and passes through the location of the first marker, on the preview image;
displaying a third vertical line on the preview image, which is parallel to a vertical axis of the preview image and passes through the location of the first marker, on the preview image; and
displaying predetermined icons at the location of the second marker, the location of the third marker, and the location of the fourth marker on the preview image.
